(19) United States
(12) Patent Application Publication
(10) Pub. No.: US 2024/0209200 A1
(43) Pub. Date: Jun. 27, 2024

(54) ULTRA-STRONG HYDROGEL FIBERS

(71) Applicant: UTI Limited Partnership, Calgary (CA)

(72) Inventors: Qingye LU, Calgary, CA (US); Xiao HE, Calgary (CA)

(21) Appl. No.: 18/288,239

(22) PCT Filed: Apr. 28, 2022
(86) PCT No.: PCT/CA2022/050647

§ 371 Date: Oct. 25, 2023

Related U.S. Application Data
(60) Provisional application No. 63/181,442, filed on Apr. 29, 2021.

(51) Int. Cl. C08L 33/26; A61L 27/44; A61L 27/52; C08J 3/075; C08K 3/08; C08K 3/30; C08K 5/092; C08K 5/13

(52) U.S. Cl. CPC C08L 33/26; A61L 27/446; A61L 27/52; C08J 3/075; C08K 3/08; C08K 3/30; C08K 5/092; C08K 5/13; A61L 2400/12; C08K 2003/0806; C08K 2003/3054; C08K 2201/005; C08K 2201/011

(57) ABSTRACT

Disclosed herein are hydrogel precursor solutions, hydrogels, and methods of preparation and uses of the same. The hydrogels may be gelled at room temperature, in the absence of added light or heat, to yield ultra-strong hydrogel fibers suitable for load-bearing applications, or as adhesives or coatings. The hydrogels may include a polymerized polymer containing acrylic acid, additional acrylic acid, an organic acid such as citric acid, and an oxidizing agent such as a persulfate salt. Silver-lignin nanoparticle suspensions may be used to initiate a free radical oxidative decarboxylation reaction in the disclosed compositions. Hydrogels may be prepared from such compositions through incubation leading to gelling. The gelled hydrogels may be stretched or spun into hydrogel fibers having desirable mechanical properties, such as strength, stretchability, and adhesion.

### CROSS REFERENCE TO RELATED APPLICATION

This application claims priority to United States Provisional Patent Application U.S. 63/181,442, filed Apr. 29, 2021, the entire contents of which is hereby incorporated by reference.

### FIELD

The present disclosure relates generally to hydrogel materials with desirable mechanical properties.

### BACKGROUND

Hydrogels have drawn a wide range of interest for constructing functional materials. For example, hydrogels with biocompatibility and antimicrobial activity may be used in biomaterials for biomedical and biological applications such as flexible electronic devices, wound repair, tissue-mimicking, and soft robots. To fulfill the diverse requirements depending on various applications, tremendous efforts are underway to construct hydrogel materials into three-dimensional (3D), two-dimensional (2D), or one-dimensional (1D) fiber-like configurations. Compared with 3D and 2D hydrogels, 1D macroscopic hydrogel fibers possess smaller cross-sectional areas, which means such thin hydrogel fibers would bear more significant tensile actuation under the same load, leading to a higher standard on the mechanical properties for engineering 1D macroscopic hydrogel fibers.

Typically, for bio-applications such as tissue-mimicking and wound repair, it is more favorable to use hydrogels with similar or superior mechanical properties compared with human tissues to better mimic, integrate or substitute the target tissues completely or in part. However, inferior mechanical properties including low tensile stress, poor stretchability, substandard modulus, and weak toughness during the load-bearing process critically constrain the range of their practical uses and, therefore, have spurred researchers to combine covalent bonds and sacrificial or reversible bonds, including hydrogen bonds, hydrophobic interactions, ionic bonds and host-guest interactions to strengthen brittle and weak networks, leading to flexible and strong hydrogels that handle energy dissipation under large deformations.

With the synergistic effect of covalent and reversible bonds, a variety of 1D macroscopic hydrogel fibers with great mechanical properties have been developed, such as artificial spider silk with twisted core-sheath hydrogel fibers (tensile strength of 895 MPa and strain of 44.3%), [Dou et al. Nat. Commun. 2019, 10, 1] ultra-stretchable fibers (tensile strength of 5.6 MPa and strain of 1,180%), [Zhao et al. Nat. Commun. 2018, 9, 1] and supramolecular fibers (tensile strength of 193 MPa and strain of 36%). [Wu et al. Proc. Natl. Acad. Sci. U.S.A 2017, 114, 8163] However, compared with specialized biomaterials possessing inherent biocompatibility, biosafety, and antimicrobial activity, the reported fibers show relatively limited potentials for biomedical and biological applications due to the missing of these intrinsic bio-properties. While hydrogel fibers that have been developed for biological and biomedical applications such as bacterial cellulose reinforced chitin fibers (tensile stress of 186.2 MPa and a strain of 8.3%), [Wu et al. Carbohydr. Polym. 2018, 180, 304.] lotus-fiber-like spiral hydrogel bacterial cellulose fibers (tensile stress of 90 MPa and strain of 290%), [Guan et al. Nano Lett. 2021] and self-helical fiber (tensile stress of 1 MPa and strain of 150%) [Wang et al. ACS Nano 2015, 9, 9167] are not strong enough when compared with the tough tissues such as muscle and tendon, confining their applications in mimicking, repairing and integrating with strong human tissues. Furthermore, many reported hydrogel fibers applied as potential structural biomaterials exhibit unsatisfactory tissue or cell adhesion, hindering the integration and unity between fibers and tissues. Based on these reasons, the design and preparation of hydrogel fibers with bio-properties, tissue or cell adhesion, and excellent mechanical properties are a promising direction for tissue-related hydrogel applications.

### SUMMARY

In one aspect there is a composition comprising: a nanoparticle (NP) suspension; an organic acid, the organic acid having carboxylate groups; an oxidizing agent; acrylic acid (AA); and a polymerized copolymer containing AA.

In one example, the NP suspension is a suspension of nanoparticles selected from the group consisting of silver, iron, copper, zinc, nickel, and combinations thereof.

In one example, the NP suspension is a suspension of silver nanoparticles.

In one example, the NP suspension comprises a phenolic compound.

In one example, the phenolic compound comprises catechol.

In one example, the NP suspension comprises Ag-lignin NPs.

In one example, the organic acid is selected from the group consisting of citric acid, malic acid, tartaric acid, and combinations thereof.

In one example, the organic acid comprises citric acid (CA).

In one example, the polymerized copolymer is selected from the group consisting of poly(acrylamide-co-acrylic acid) (P(AAm-co-AA)), poly (ethylene glycol) dimethacrylate (PEGDA), polyacrylic acid (PAA), and combinations thereof.

In one example, the polymerized copolymer comprises poly(acrylamide-co-acrylic acid) (P(AAm-co-AA)).

In one example, the composition is an aqueous solution.

In one example, the oxidizing agent is selected from the group consisting of ammonium persulfate, potassium persulfate, hydrogen peroxide, a ferric solution, and combinations thereof.

In one example, the oxidizing agent comprises ammonium persulfate.

In one example, the NP suspension is an Ag-lignin NP suspension obtained by a redox reaction between [Ag(NH3)2]+ and lignin aqueous solution.

In one example, the composition comprises the NP suspension in an amount of about 20 v/v % to about 60 v/v %, such as about 40 v/v %.

In one example, the composition comprises the organic acid in an amount of from about 0.5 v/v % to about 2.5 v/v %, such as about 2.0 v/v %.

In one example, the composition comprises the AA in an amount of about 10 v/v % to about 20 v/v %, such as about 15 v/v %.

In one example, the composition comprises the polymerized copolymer in an amount of from about 0.1 w/v % to about 20 w/v %, such as about 10 w/v %.

In one example, the composition further comprises an additive selected from the group consisting of anti-odor agents, antimicrobial agents, antioxidants, plasticisers, colorants, other polymers such as conductive polymers, metal/metal oxide nanoparticles, and any combination thereof.

In one aspect there is a hydrogel obtained by gelling the composition according to any one of the preceding examples.

In one example, the hydrogel is obtained by incubating the composition at room temperature for an amount of time of about 8 hours to about 48 hours, or about 16 hour to about 24 hours, such as about 8 hours, about 16 hours, or about 24 hours.

In one example, the hydrogel is obtained by gelling the composition in the absence of UV radiation or heating.

In one example, the hydrogel has tensile stress in the range of about 97.6 MPa to about 422.0 MPa.

In one example, the hydrogel has strain in the range of about 6.7% to about 95.2%.

In one example, the hydrogel has Young's modulus in the range of about 1.2 GPa to about 8.7 GPa.

In one example, the hydrogel has toughness in the range of about 3.9 MJ m-3 to about 281.6 MJ m-3.

In one example, the hydrogel has antimicrobial activity, such as antibacterial activity.

In one example, the hydrogel is a hydrogel fiber.

In one example, the hydrogel fiber is micro-sized, having a diameter in the range of about 20 μm to about 150 μm.

In one aspect, there is use of the hydrogel according to any one of the preceding examples, for bearing a load.

In one aspect, there is use of the hydrogel according to any one of the preceding examples, as an adhesive or a coating.

In one example, the hydrogel is for adhesion to, or to coat: mammalian tissue such as skin, organs, bone, kidney, heart, lung, or liver; plant or plant materials such as fruits or vegetables; or other materials such as polymers, rubbers, metals, fibers, or clothing such as masks.

In one aspect, there is use of the hydrogel according to any one of the preceding examples, in a biomedical application or device.

In one example, the biomedical application or device is selected from the group consisting of medical devices, implants, wound repair, sutures, bandages, coatings, and artificial tissues.

In one aspect, there is an artificial tissue comprising the hydrogel according to any one of the preceding examples.

In one example, the artificial tissue tissue is muscle, tendon, or cartilage.

In one aspect, there is a method of preparing a hydrogel comprising: combining nanoparticles, an organic acid, an oxidizing agent, acrylic acid (AA), and a polymerized copolymer containing AA, in an aqueous solution to form a hydrogel precursor solution; and incubating the hydrogel precursor solution to form the hydrogel.

In one example, the nanoparticles comprise silver, iron, copper, zinc, or nickel nanoparticles.

In one example, the nanoparticles comprise a suspension of Ag-lignin NPs.

In one example, the organic acid comprises citric acid.

In one example, the oxidizing agent comprises a persulfate salt.

In one example, the polymerized copolymer comprises poly(acrylamide-co-acrylic acid) (P(AAm-co-AA)).

In one example, the combining step comprises combining the polymerized copolymer with one or more solutions comprising the AA, the organic acid, the nanoparticles, the oxidizing agent, or any combination thereof.

In one example, incubating the hydrogel precursor solution comprises incubating at room temperature.

In one example, incubating the hydrogel precursor solution comprises incubating for an amount of time in the range from about 8 hours to about 48 hours.

In one example, the method further comprises preparing Ag-lignin NPs prior to the combining step.

In one example, the method further comprises stretching the formed hydrogel to form a hydrogel fiber.

In one example, stretching the formed hydrogel comprises a spinning process.

In one example, the method further comprises agitation of the precursor solution, such as mechanical agitation or sonication.

In one example, the incubating step is in the absence of UV radiation or heating.

### BRIEF DESCRIPTION OF THE INVENTION

Embodiments of the present disclosure will now be described, by way of example only, with reference to the attached Figures.

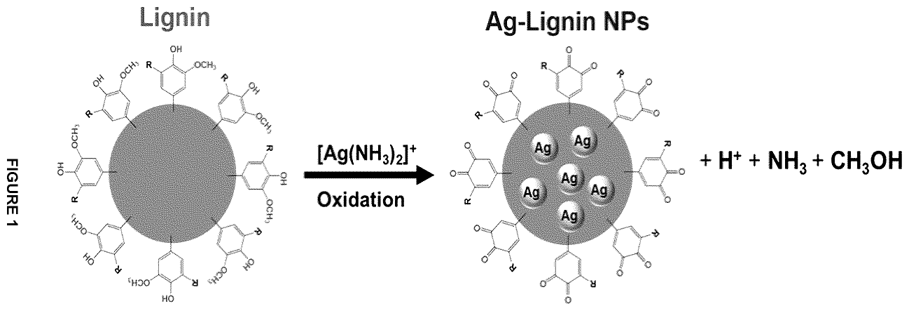

FIG. 1 shows a synthetic route to Ag-lignin nanoparticles by a redox reaction between reductive lignin and [Ag(NH₃)₂]⁺,

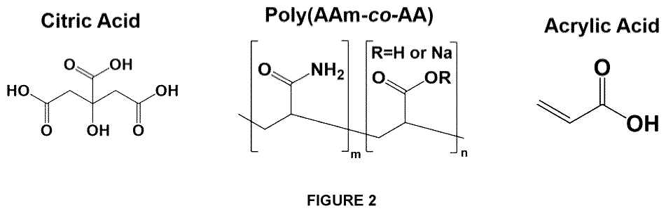

FIG. 2 shows the molecular structures of citric acid, P(AAm-co-AA), and acrylic acid.

FIG. 3 shows a schematic illustration of a synthesis of ultra-stretchable hydrogel with two combined catalysis reactions: oxidative decarboxylation and quinone-catechol reversible redox reaction.

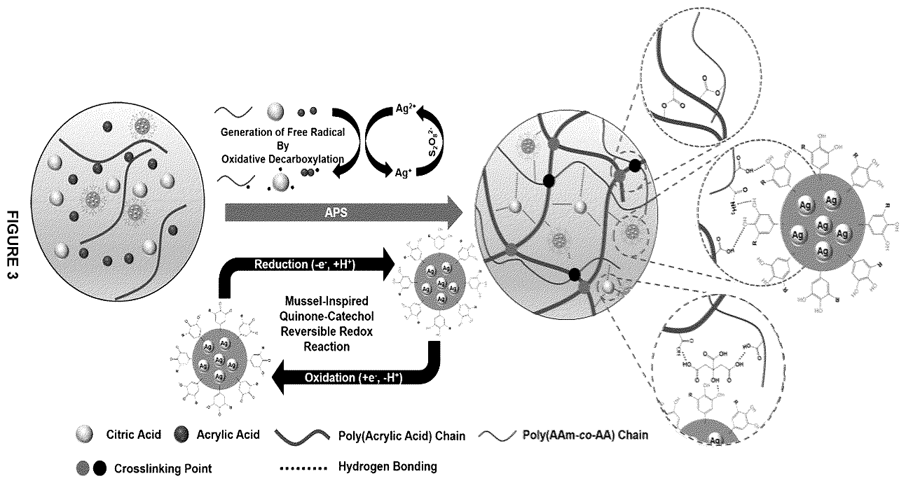

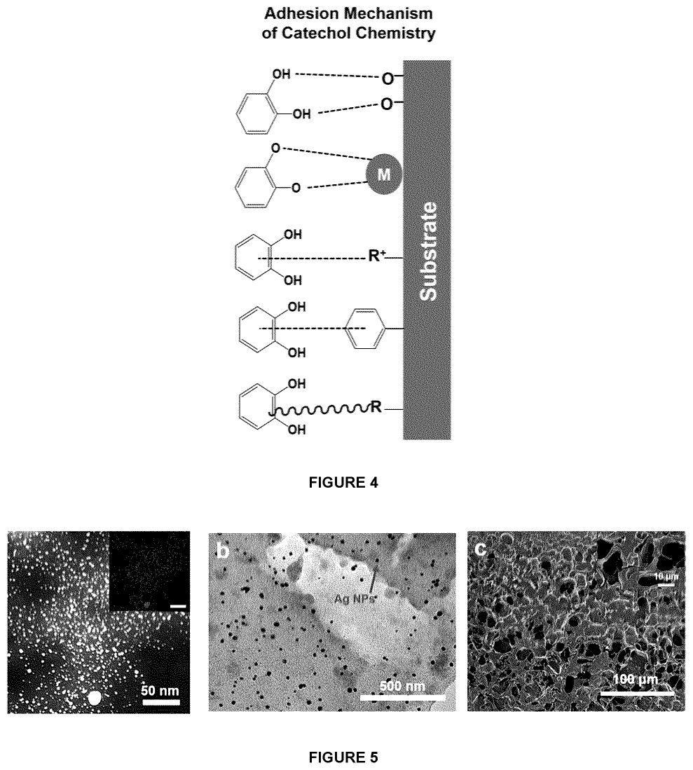

FIG. 4 shows an illustration of the adhesion mechanism of catechol groups by interacting with the substrate via hydrogen bonding, coordination bonding, cation-π bonding, π-π interaction and/or covalent linking (respectively, from top to bottom).

FIG. 5 shows (a) a high-angle annular dark-field scanning transmission electron microscopy (HAADF-STEM) image of exemplary Ag-lignin NPs, and the inset with a scale bar of 50 nm shows the corresponding EDX mapping of Ag, confirming the presence of Ag NPs in the exemplary Ag-lignin NPs; (b) a transmission electron microscopy (TEM) image of exemplary Ag-lignin NPs, confirming the uniform distribution of Ag NPs in the Ag-lignin NPs; and (c) the microstructure of an exemplary (0.5P-1AA-0CA) hydrogel.

FIG. 6 shows (a) tensile stress-strain of an exemplary hydrogel synthesized with different volume ratios of AA/CA solution, and (b) images of manually stretching of a small tip of 0.5P-1AA-2.5CA hydrogel (thickness ˜5 mm) to 1,300 mm.

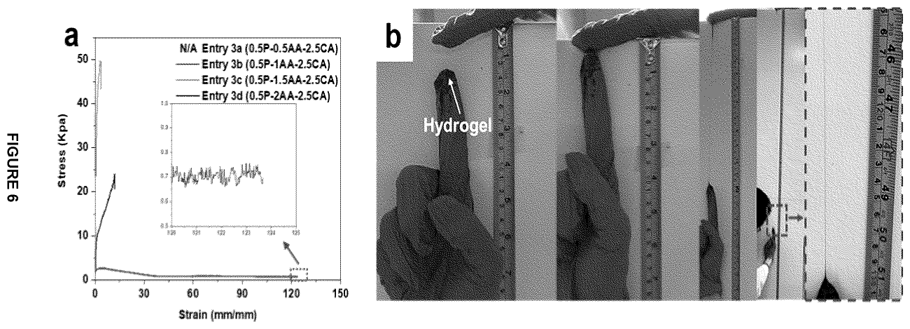

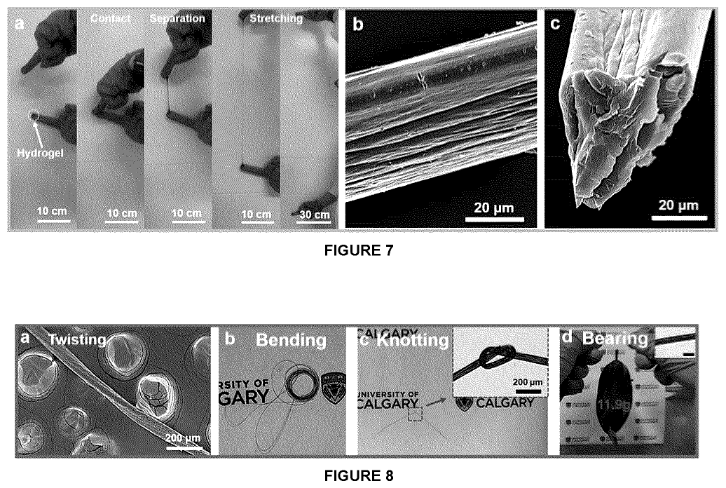

FIG. 7 shows (a) images of manual fabrication of a hydrogel fiber from ultra-stretchable hydrogel through a “Contact-Separation-Stretching” method, and scanning electron microscopy (SEM) images of the (b) external part, and (c) cross-section of the hydrogel fiber.

FIG. 8 shows images demonstrating the ability of exemplary hydrogel fibers to withstand (a) twisting, (b) bending, (c) knotting, and (d) lifting an ornament (11.9 g) using a single hydrogel fiber with a diameter of ˜50 μm.

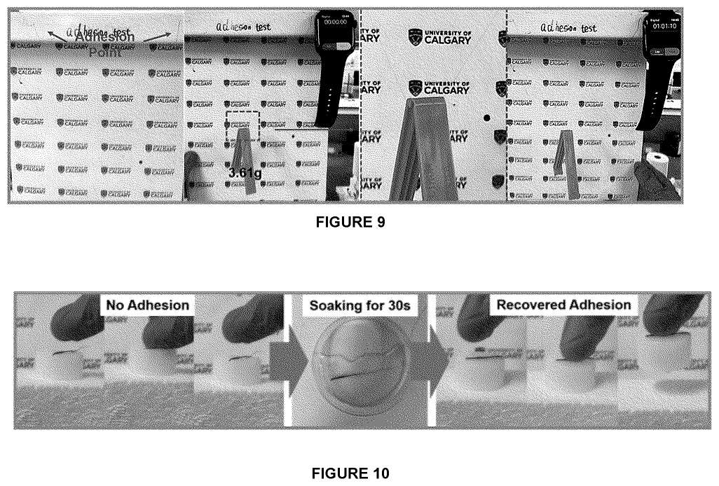

FIG. 9 shows images demonstrating that an exemplary hydrogel fiber can be adhered to a metal shelf while holding a lab clamp (3.61 g) for 1 h.

FIG. 10 shows images demonstrating the recovered adhesion of a dried hydrogel fiber after soaking in DI water for 30 s.

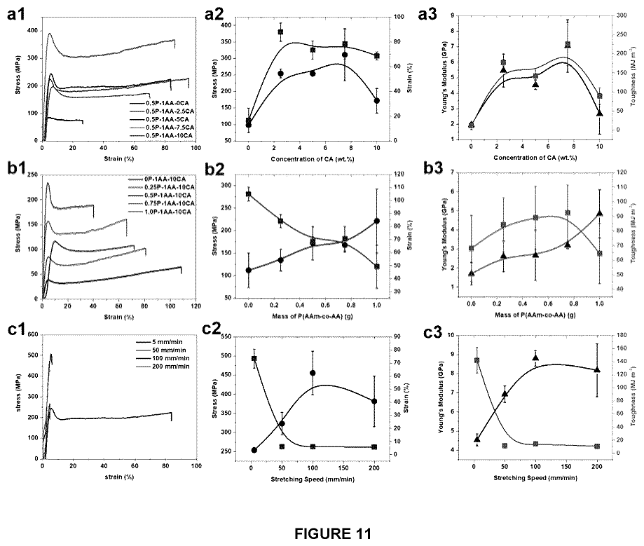

FIG. 11 shows the (1) tensile stress-strain curves, (2) max stress and breaking strain, or (3) Young's modulus in low strain region and corresponding toughness as a function of (a) weight percentage of CA, (b) mass of P(AAm-co-AA), or (c) stretching speed. Exemplary 0.5P-1AA-5CA fibers were used for the measurements regarding the stretching speed.

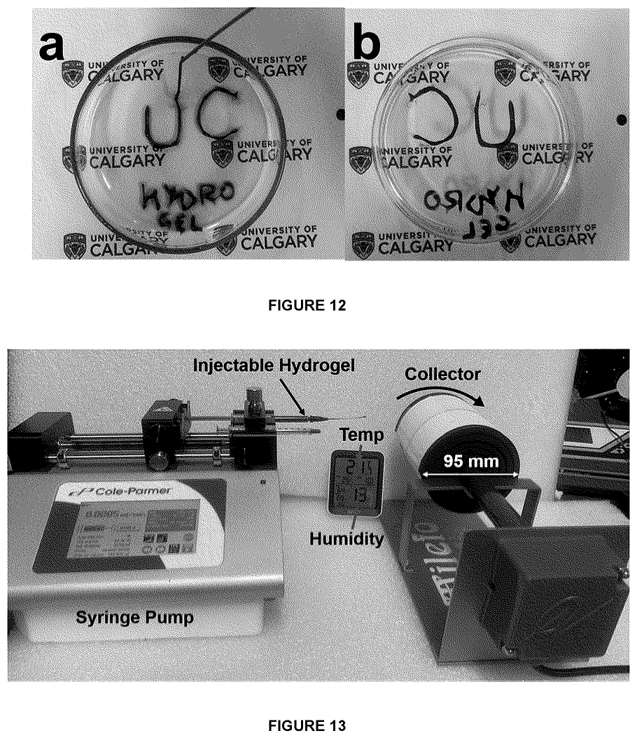

FIG. 12 shows images demonstrating (a) “UC HYDROGEL” written by the hydrogel injected through a 19 gauge needle, and (b) the written words adhered onto an upturned glass peri dish, indicating the strong adhesion of the exemplary injected hydrogel.

FIG. 13 shows an example of a spinning apparatus for the scalable production of hydrogel fibers according to one or more embodiments of the present disclosure.

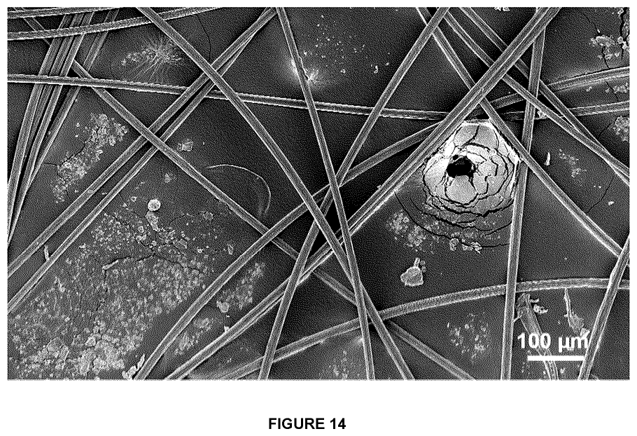

FIG. 14 shows a microscopic photo of the as-spined fibers with a uniform diameter of ˜20 μm.

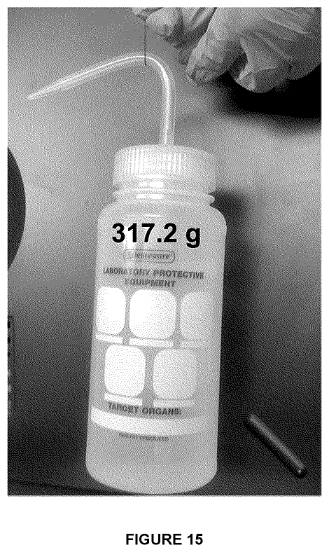

FIG. 15 shows an image demonstrating that lifting bottled methanol (317.2 g) using piled spined hydrogel fibers (9.5 mg), demonstrating the excellent mechanical property of the exemplary spined fibers.

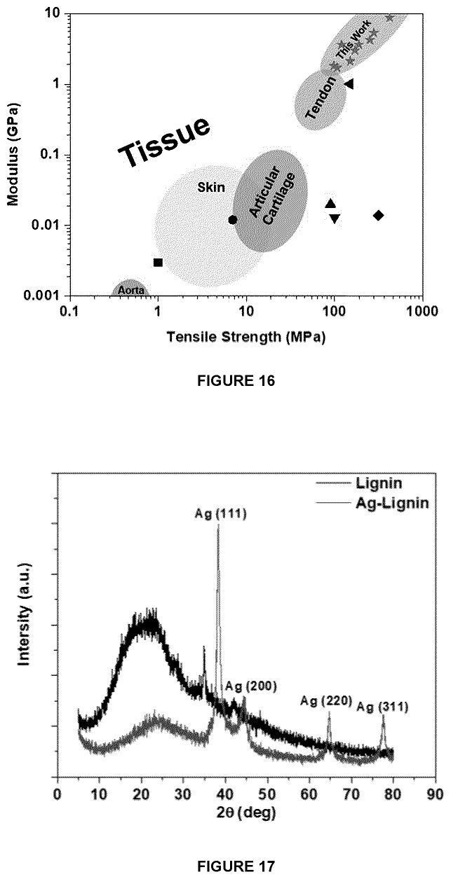

FIG. 16 shows the Ashby plot of modulus versus tensile strength of human tissues, reported hydrogel fibers as biomaterials, and the exemplary fibers disclosed herein.

FIG. 17 shows XRD patterns for exemplary Ag-lignin NPs and lignin NPs.

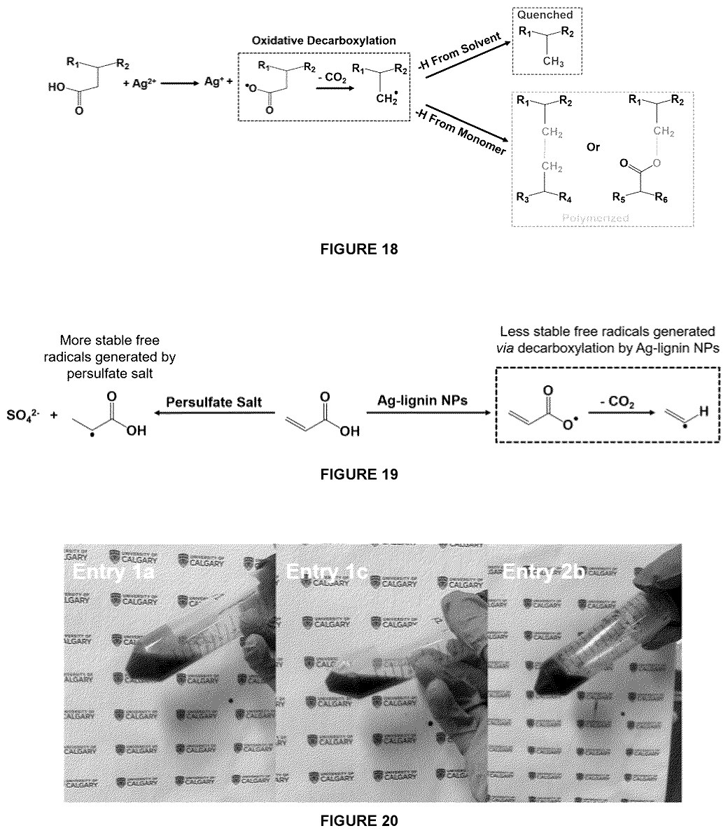

FIG. 18 shows a mechanism of polymerization induced by a decarboxylation reaction of monomers containing —COOH groups (R1-6 are the different monomers).

FIG. 19 shows generation of different free radicals from acrylic acid monomer by persulfate salt and Ag-lignin NPs.

FIG. 20 shows a less desirable gelation of precursor hydrogel solutions without AA.

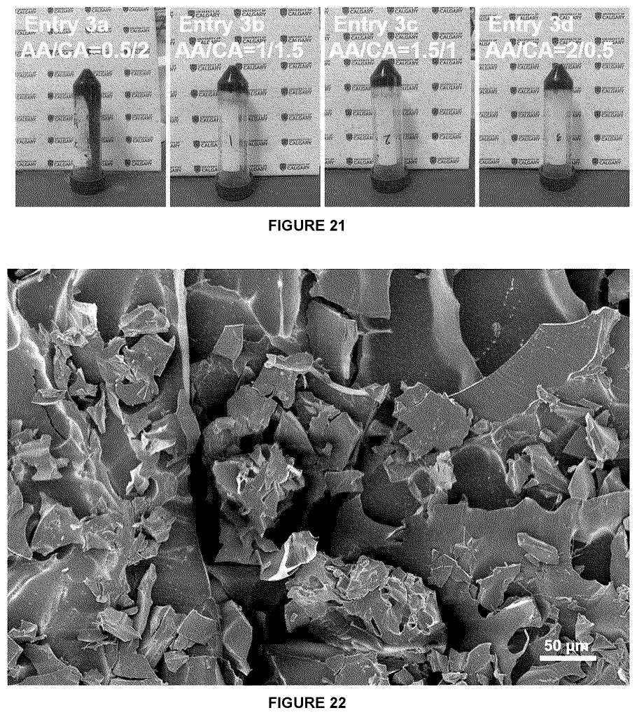

FIG. 21 shows gelation of hydrogel solutions with different volume ratios of AA/CA solution.

FIG. 22 shows a SEM image of the exemplary 0.5P-1AA-7.5CA hydrogel, showing the compact and surprising porous structure

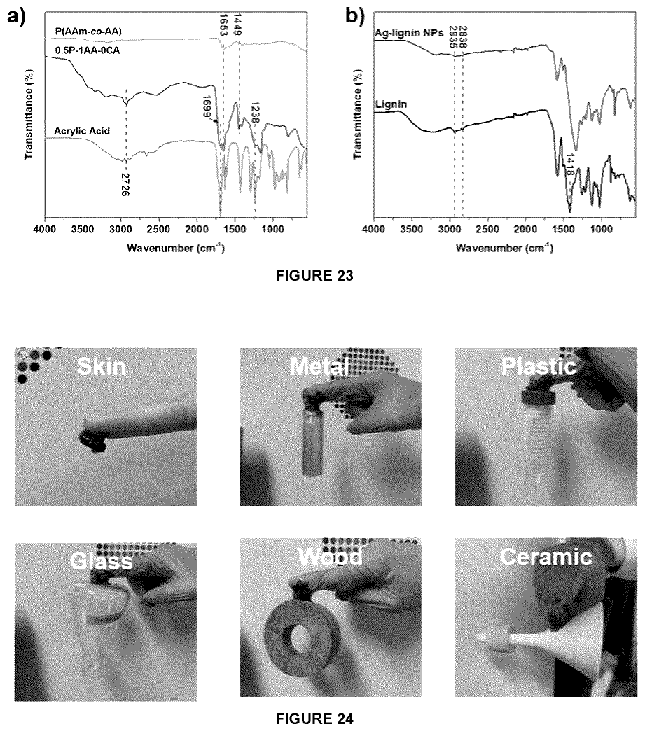

FIG. 23A&B shows the spectra of Ag-lignin NPs, lignin, P(AAm-co-AA), acrylic acid, and 0.5P-1AA-OCA hydrogel.

FIG. 24 shows the robust adhesion properties of the synthesized hydrogel on different surfaces.

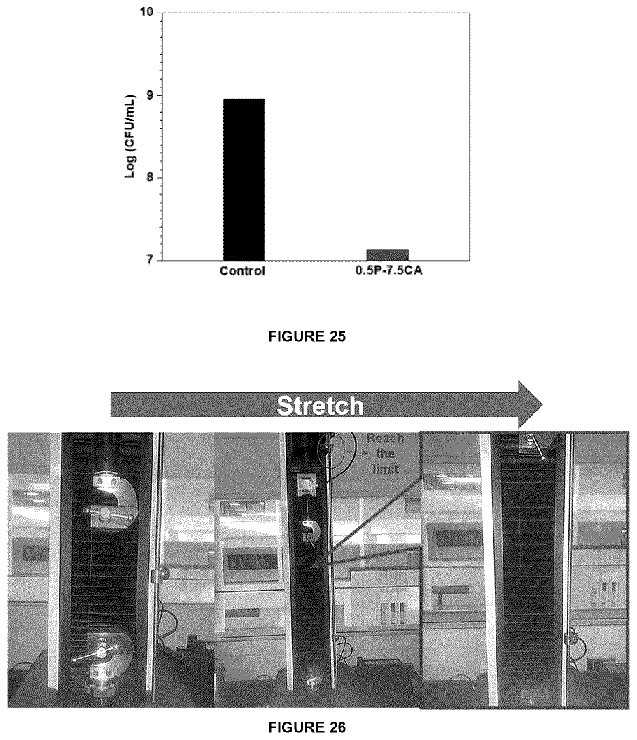

FIG. 25 shows the number of CFUs of E. coli in LB broth after 24 h incubation without or with 10 mg/mL 0.5P-1AA-7.5CA hydrogel.

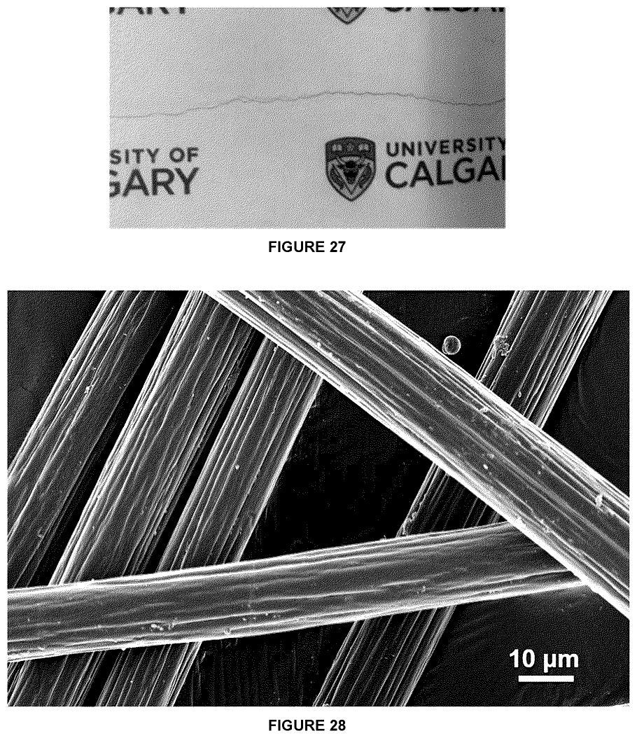

FIG. 26 shows an exemplary synthesized hydrogel (Entry 3b, 0.5P-1AA-2.5CA) under full stretching using a universal testing machine.

FIG. 27 shows a hydrogel fiber with wrinkles obtained via a “Contact-Separation-Stretching” method, followed by an immediate release.

FIG. 28 shows a SEM image of hydrogel fibers obtained from a spinning process, illustrating the paralleled alignments on the fiber surfaces.

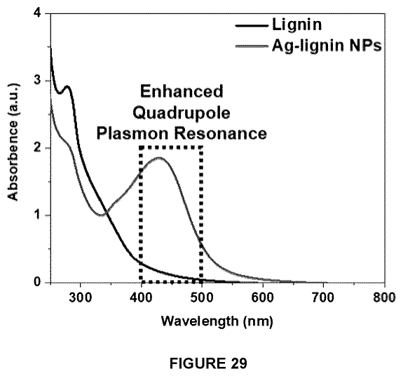

FIG. 29 shows the UV-vis spectrum of lignin and Ag-lignin NPs, illustrating the enhanced quadrupole plason resonace in the region of 400 nm to 500 nm with the presence of silver NPs.

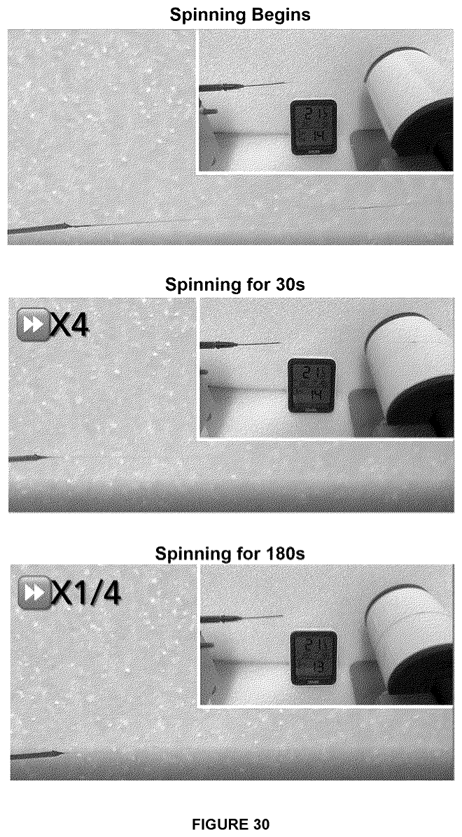

FIG. 30 shows an exemplary continuous spinning process to produce hydrogel fibers.

### DETAILED DESCRIPTION

Recently, Ag-lignin nanoparticle (NP) based hydrogels were designed based on oxidative decarboxylation by the reaction between silver-lignin nanoparticles (Ag-lignin NPs) and persulfate salt, [Afewerki et al. ACS Nano 2020; Weng et al. Soft Matter 2017, 13, 5028] leading to successful gelation without the need to use external stimuli such as ultraviolet (UV) irradiation or heating, which may be harmful to tissues and cells. It is suspected that the long-lasting adhesion induced by the quinone-catechol reversible redox reaction in the Ag-lignin NPs, together with the strong antimicrobial activity resulting from released silver ions (Ag⁺), provides Ag-lignin NP based hydrogels with promising potential for use in biomedical science, such as tissue engineering. Despite such promising bio-properties, Ag-lignin NP based hydrogels still show limited mechanical properties (tensile stress of 120 kPa and strain of 2,750%), [Gan et al. Nat. Commun. 2019, 10, 1] which severely constrains their applications towards strong and tough tissues. Moreover, limited stretchability is the major hurdle for generating the corresponding 1D macroscopic hydrogel fiber by drawing methods. Nonetheless, the present disclosure shows that, by incorporating plentiful reversible bonds, the resulting Ag-lignin NP-based hydrogels exhibit much better stretchability to enable effective fabrication of desirable 1D macroscopic hydrogel fibers with inherent bio-properties and excellent mechanical properties. Such materials may be useful in any suitable load-bearing application, or for use as an adhesive or coating. For example, given their inherent biocompatibility and antimicrobial activity, such materials may be useful in biological and biomedical applications.

Generally, the present disclosure provides a hydrogel with impressive stretchability, long-term adhesion, and strong antimicrobial activity. Such a hydrogel may be used for fabricating ultra-strong 1D macroscopic hydrogel fibers. The hydrogel may be gelled from the precursor solution containing Ag-lignin NPs, citric acid (CA), acrylic acid (AA), and poly (acrylamide-co-acrylic acid) (P(AAm-co-AA)) without UV radiation or heating. Gelling in the absence of UV radiation or heating may be beneficial for avoiding potentially harmful treatment to tissues. It is suspected that the interpenetrating network together with plentiful hydrogen bonds formed between the carboxylate groups and hydroxyl groups of these materials are responsible for enormous energy dissipation under extensive deformation, leading to the ultra-high stretchability (e.g. over 12,400%) of the hydrogel. Hydrogel materials prepared under the disclosed conditions possessed promising bio-properties, including long-term adhesion and strong antimicrobial activity, due to the quinone-catechol reversible redox reaction and released Ag⁺ by the Ag-lignin NPs.

As disclosed herein, a strong hydrogel fiber with a micro-sized diameter (˜50 μm) could be facilely obtained via manual stretching, and the as-generated fiber exhibits excellent mechanical properties (tensile stress of 422.0 MPa, strain of 86.5%, Young's modulus of 8.7 GPa, and toughness of 281.6 MJ m-3). In addition, the mechanical properties could be adjusted over a wide range by varying the dosage of CA and P(AAm-co-AA) in the precursor solution, and the resulting mechanical properties are comparable or even stronger when compared with human tissues. Together, this suggests the hydrogel may have wide adaptability and potential in suitable applications, such as tissue engineering. Moreover, the hydrogel fiber may be simple to manufacture. For example, the hydrogel fiber could be produced on a large scale using a simple house-built spinner while maintaining excellent mechanical properties. As disclosed herein, an exemplary hydrogel was capable of lifting a load that was ˜33,000 times heavier than the applied fibers. Hydrogels of the present disclosure may have inherent antimicrobial activity, adhesion, excellent mechanical properties, and amenability to scalable production. With these properties, it is expected that such 1D microscopic hydrogel fibers may be suitable for use in wound repair, tissue-mimicking and integration, and soft robots in biological and biomedical science, for example.

FIGS. 1-4 illustrate the general procedures and molecular structures of the raw materials for preparing a hydrogel with ultrahigh stretchability, long-term adhesion, and strong antimicrobial activity, according to one or more embodiments of the present disclosure. Ag-lignin NPs may be prepared via the redox reaction between the [Ag(NH₃)₂]⁺ and lignin-dissolved aqueous solution. During such a reaction, the functional groups, such as methoxy groups and phenolic hydroxyls in the lignin, are oxidized into the corresponding quinone/semiquinone groups. Meanwhile, the silver ions are reduced to metallic silver NPs, which may be well dispersed in the Ag-lignin nanoparticles, as demonstrated in the example of FIG. 5. As shown in the associated XRD patterns (FIG. 17), the pattern of lignin in that example showed a broad peak, while the characteristic peaks at 2θ=38.3°, 44.2°, 64.4°, and 78.1° were observed in the pattern of Ag-lignin NPs, corresponding to the (111), (200), (220) and (311) planes of the metallic silver NPs, respectively. This further confirmed the reduction of Ag⁺ to Ag in the Ag-lignin NPs of that example. The Ag-lignin NPs obtained as such influence the subsequent gelation. The Ag-lignin NPs are suggested to have a role in the generation of free radical from monomers by oxidative decarboxylation to initialize the polymerization. The Ag-lignin NPs are suggested to have a role in the creation of long-lasting quinone-catechol reversible redox reactions, to help realize long-term adhesion of the hydrogel.

As schematically illustrated in FIG. 3, the Ag⁺ and photo-generated electron may be formed due to the surface plasmon resonance of metallic Ag NPs under visible light and/or UV light. The Ag⁺ generated as such may be oxidized to Ag²⁺ by persulfate salt in the precursor solution, and the Ag²⁺, with strong oxidizing ability, may eventually enable the oxidative decarboxylation of carboxylate groups in the monomers to generate free radicals (FIG. 18). It is suspected that both the free radicals generated by oxidative decarboxylation, together with the free radicals generated from the broken C═C bonds induced by the initiator (persulfate salt), contribute to subsequent gelation at room temperature (FIG. 19). In the meantime, the photo-generated electrons from Ag-lignin NPs may convert the quinone and/or hydroquinone groups in Ag-lignin NPs into catechol groups, resulting in abundant catechol chemistry on the Ag-lignin nanoparticle surfaces. Thus, the continuously photo-generated electrons could affect persistent production of catechol groups in the Ag-lignin NPs, realizing a quinone-catechol reversible redox reaction and resulting in long-term adhesion despite oxidation by oxygen in the air. Without wishing to be bound by any particular theory, it is suspected that the synergistic effect of free radicals generated by Ag-lignin NPs and persulfate salt, as well as the long-term quinone-catechol reversible redox reaction, results in the gelation of hydrogel solutions according to embodiments of the present disclosure.

In the disclosed exemplary embodiments, AA, P(AAm-co-AA), a CA aqueous solution and/or an Ag-lignin NP solution were mixed at room temperature to investigate possible gelation phenomena. As shown in Table 1, the gelling phenomena were less desirable for entries without AA (Entries 1a, 1c, and 2a, FIG. 20), indicating that polymerization by the free radicals generated via oxidative decarboxylation alone could not result in a complete gelation process. It is suspected that this is because the alkyl free radicals generated as such are not stable and are easily quenched by abstracting a hydrogen atom from the solvent (FIGS. 18 and 19). Besides the effect of introducing AA to the precursor solution, the volume ratio of AA/CA solution also influenced gelation. For the precursor solutions disclosed herein (Table 1, Entries 3a-3d), gelling was more desirable when the ratio was more than 1:4 (FIG. 21). This demonstrates the effect of polymerization by free radicals generated from the broken C═C bonds by the persulfate salt (FIG. 19).

The volume ratio between AA and CA in the aqueous solution is explored in the exemplified embodiments. A gelled 0.5P-1AA-OCA hydrogel showed the structure shown in FIG. 5, which appears relatively porous. A gelled 0.5P-1AA-7.5CA hydrogel presented a more compact and less porous structure (FIG. 22) at the same magnification. It is suspected that this may be due to increased crosslinking density. Increased crosslinking density may be induced by the higher concentration of CA in the precursor solution. Increased crosslinking density may contribute to the improved mechanical properties of the generated hydrogel fibers exemplified herein.

Exemplary FTIR spectra of lignin, Ag-Lignin NPs, Ag-lignin NP-based hydrogels, and raw materials including P(AAm-co-AA) and AA are shown in FIG. 23. In the lignin spectrum, the peaks at ˜2935, ˜2838, and ˜1418 cm⁻¹ were attributed to the methyl and methylene groups, symmetric stretch for —CH₃ in the methoxy groups, and C—O stretching vibration, respectively. However, such peaks decreased in intensity or disappeared in the spectrum of the exemplified Ag-lignin NPs, indicating the decrease or removal of methoxy groups from the lignin, resulting from oxidation by [Ag(NH₃)₂]⁺ during the synthesis of Ag-lignin NPs. The peaks at ˜2726, 1699, and 1238 cm⁻¹, ascribed to the C—H groups, C═O groups, and O—H bending of AA, respectively, were observed in the spectrum hydrogel, indicating the existence of AA in the final hydrogel product. The peaks at 1653 cm⁻¹ and 1449 cm⁻¹ resulting from the C═O group stretching and CH₂ bending, respectively, confirmed the presence of P(AAm-co-AA) in the hydrogel.

The exemplary hydrogels exhibited robust adhesion to various surfaces such as human skin, metal, plastic, glass, wood, and ceramic (FIG. 24). It is suspected that this results from the plentiful carboxylate and catechol groups in the hydrogel. For the adhesion mechanism, the carboxylate groups contribute to the formation of electrostatic interactions between the hydrogel and various surfaces. It is also suspected that the catechol groups could interact with specific substrates containing amine or thiol groups via covalent bonding by Michael addition and/or Schiff base reactions, or form reversible interactions such as hydrogen bonding, coordination bonding, cation-π bonding, π-π interactions with regular substrates (FIG. 4). Therefore, it is expected that the adhesion properties of hydrogels according to the present disclosure could be human skin, human tissues and organs such as bone, kidney, heart, lung, liver, etc, and other common materials in the daily life, including rubber, polypropylene, polytetrafluoroethylene, vegetables and fruit.

Ag NPs in the hydrogel according to embodiments of the present disclosure may release Ag⁺ ions. Accordingly, it is expected that such hydrogels may display antimicrobial activity. This is demonstrated for an exemplary hydrogel in FIG. 25, which shows a 1.83-log reduction of CFU as compared to a control sample. Therefore, it is expected that the hydrogels disclosed herein may be useful in applications where antimicrobial activity is beneficial. The hydrogels may be used in bio-applications, such as implants, wound repair, adhesive bandage, mask coating, anti-infection coating, and medical devices.

FIG. 6 shows the typical tensile stress-strain curves of some exemplary hydrogels synthesized from different precursor solutions (Table 1, Entries 3b-3d). With the higher volume ratio of AA/CA solution, the hydrogels (0.5P-1.5AA-2.5CA and 0.5P-2AA-2.5CA) exhibit higher tensile stress and lower strain. Without wishing to be bound by any particular theory, it is suspected this is a result of higher density crosslinking by the covalent bonding, which makes the hydrogel more rigid and less stretchable. Surprisingly, the hydrogel of Entry 3b (0.5P-1AA-2.5CA) with a volume ratio of AA/CA solution for 1:1.5 showed impressive stretchability (over 12,400%) and did not break even when the universal machine reached the operation limit (FIG. 26). Such impressive stretchability is most likely attributable to a synergistic effect of factors, which may include the abundant and homogeneously distributed hydrogen bonds, the well-dispersed Ag-lignin NPs, and the interpenetrated networks throughout the hydrogel of such an embodiment. The long P(AAm-co-AA) chains with high molecular weight may interpenetrate the formed PAA network in an exemplary hydrogel, which may strengthen the hydrogel undergoing the stretching process. Due to their hydrophilicity, it is expected that the Ag-lignin NPs disperse well in the hydrogel and may act as homogeneously distributed centers to connect nearby components via plentiful hydrogen bonds among hydroxyl and carboxylate groups. Furthermore, the P(AAm-co-AA) chains, AA, CA, and Ag-lignin NPs in the exemplified precursor solutions contain plentiful functional groups such as hydroxyl, carboxylate, and amine groups, which may favor the formation of abundant and uniform hydrogen bonding within a hydrogel formed therefrom. The well-dispersed Ag-lignin NPs may also improve the mechanical properties of the hydrogel because of the nano-reinforcement effect. Therefore, the plentiful and homogeneously distributed hydrogen bonds, the well-dispersed Ag-lignin NPs, as well as the interpenetrated network throughout the whole hydrogel contribute to dissipating enormous energy under large deformation, leading to excellent stretchability. To further illustrate the potential properties of the disclosed compositions, such as excellent stretchability, a small tip of an exemplary (0.5P-1AA-2.5CA) hydrogel (thickness: ˜5 mm) was manually stretched to ˜1,300 mm, demonstrating an ultimate strain even higher than 26,000% (FIG. 7).

It will be understood that the hydrogels, precursor solutions, and compositions disclosed herein may be tuned to various purposes and applications. For example, the ratio of components, such as the volume ratio of AA/CA, could be tuned to impart different properties on the resultant hydrogel. For exemplary purposes, a precursor solution with a volume ratio (1:1.5) of AA/CA solution was gelled at room temperature and formed a hydrogel exhibiting excellent stretchability. From such a hydrogel, the subsequent fabrication of 1D macroscopic hydrogel fibers via a “Contact-Separation-Stretching” method (FIG. 7a) was possible. The just-fabricated hydrogel fiber exemplified in FIG. 7a demonstrated significant elasticity and good resilience, indicated by the immediate shrink after releasing the extension force and leading to a hydrogel fiber with many wrinkles and non-uniform diameters (FIG. 27). This observation further demonstrates the promising mechanical properties of the hydrogels disclosed herein, as the physical and chemical properties of the exemplified hydrogel resulted in the great resilience of the just-fabricated hydrogel fiber.

To obtain a straight hydrogel fiber with uniform diameter, an exemplary fabricated wet hydrogel fiber was stretched to keep straight and exposed to ambient air for 5 min. An exemplary manually-drawn hydrogel fiber obtained as such showed a uniform diameter of approximately 50 μm, and paralleled alignments were observed along the stretching direction (FIG. 7b). This was also observed on the spined hydrogel fiber surfaces (FIG. 28). A cross-section of an exemplary hydrogel fiber (FIG. 7c) exhibited obvious cracks, which may have originated from the stretched pores during the manual drawing process. Owing to the internal network constructed by covalent and hydrogen bonding, the hydrogel fiber not only has a fine diameter but also is endowed with robust mechanical properties to withstand twisting, bending, and knotting, as exemplified in FIG. 8, respectively. Impressively, an exemplary hydrogel fiber of a fine diameter ˜50 μm could hold a load of 11.9 g (FIG. 8d), demonstrating its remarkable toughness to absorb energy and plastically deform without fracture. Together with the adhesive property, likely imparted by the quinone-catechol reversible redox reaction, the tough hydrogel fiber exemplified in FIG. 9 could realize long-lasting adhesion to a metal shelf while holding a plastic clamp (3.61 g) for at least 1 h.

It is suspected that the adhesive property of hydrogel fibers may decrease after long-term exposure to ambient air. However, as shown in FIG. 10, the adhesion of a hydrogel according to an embodiment of the present disclosure could be regenerated by soaking in water for 30s, exemplifying the potential for quick recoverability of adhesion even after drying. It is suspected that this effect is due to the long-lasting quinone-catechol reversible reaction, as supported by the evidence for photo-generated electrons from Ag by quadrupole plasma resonance (FIG. 29). Furthermore, as the hydrogel may contain ionized H⁺ (FIG. 3), re-wetting the hydrogel could favor the ionization of H⁺ from CA and P(AAm-co-AA), resulting in recovered quinone-catechol reversible reaction and adhesion. Together, the exhibited inherent antimicrobial activity, strong adhesion, excellent load-bearing ability, and great toughness of the exemplary fibers fabricated from Ag-lignin NP-based hydrogels shows the great potential for the compositions and hydrogels disclosed herein. For example, these properties could be desirable in tissue integration and artificial tissue fibers in biological and biomedical science.

For tissue-related biological and biomedical applications, it may be desirable for biomaterials with adjustable and strong mechanical properties to fit and/or mimic the target tissues. Herein it is demonstrated that the hydrogels according to the invention may be suitable for such uses. For example, adjusting the dosage of P(AAm-co-AA) or CA used in the precursor solution influenced the mechanical properties, including the maximum tensile stress, ultimate strain, Young's modulus, and toughness of the hydrogel fibers. It is expected that these properties could be tuned in a wide range. As shown in FIG. 11 a1-a3, changing the dosage of CA alone could significantly strengthen the mechanical properties of an exemplary hydrogel fiber. Increasing the concentration of CA in a series of exemplary precursor solutions resulted in an initial increase in all of the measured mechanical properties of a fiber formed therefrom, with the highest strain of 95.2% at 2.5 w/v % CA (86.5% at 7.5 w/v % CA), and the highest stress of 422.0 MPa, Young's modulus of 8.7 GPa, the toughness of 281.6 MJ m⁻³ at 7.5 w/v % CA, which were higher than those of the fiber without CA (0.5P-1AA-OCA, strain: 26.5%, stress: 98.5 MPa, Young's modulus: 2.1 GPa and toughness: 18.72 MJ m⁻³). However, further increases in the CA concentration for the exemplary series resulted in a decrease in the measured mechanical properties. It is suspected that this may be due to compact crosslinking caused by increased oxidative decarboxylation of —COOH groups and hydrogen bonds, making the whole fiber more rigid and less tough.

Other adjustments to the compositions disclosed herein may result in adjustments in the properties thereof. For example, a change in the mass of P(AAm-co-AA) within a series of exemplary precursor solutions showed a linear relationship with the mechanical properties of resulting hydrogel fibers (FIG. 11 b1-b2). With the increasing mass of the P(AAm-co-AA) in the precursor solutions, it is suspected that the hydrogel may be strengthened by a more interpenetrated network. Of the measured embodiments, the resulting fiber showed the highest stress (279.8 MPa), lowest strain (38.9%), and highest Young's modulus (5.8 GPa) when a maximum amount (1 g) of P(AAm-co-AA) was used (Table 1, Entry 5d: 1P-1AA-10CA). The highest toughness measured herein was 111.8 MJ m⁻³ at 0.75 g of P(AAm-co-AA), and a further increase in the mass of P(AAm-co-AA) lowered the toughness to 41.0 MJ m⁻³. This may be because the toughness was determined by integrating the corresponding stress-strain curve, and poor stretchability of the fiber results in a weak toughness despite the highest stress obtained at that mass of P(AAm-co-AA). As the mechanical properties are influenced by the dosage of at least P(AAm-co-AA) and CA in the precursor solution, it is expected that the properties could be adjusted in a wide range depending on the application. Interestingly, hydrogel fibers, according to some exemplified embodiments, showed strain-rate-dependent mechanical properties (FIG. 11 c1-c3). The maximum stress of such fibers increased initially with increasing stretching speed and reached the highest value as 512.4 MPa at a stretching speed of 100 mm/min, and a further increase in the stretching rate led to a bit lower stress as 453.2 MPa at 200 mm/min. However, the stretchability of the exemplary fiber was almost gone, and only a strain of ˜6.0% was obtained at high stretching speeds. Similar to the change in stress and strain as a function of stretching speed, the highest Young's modulus was obtained as 9.2 GPa at a speed of 100 mm/min, and the toughness of the fiber dropped to ˜13 MJ m⁻³ with increasing stretching speed, suggesting the rupture of the hydrogel fiber at the high deformation rate. Nonetheless, the excellent mechanical properties at the normal deformation rate, antimicrobial activity, and long-term adhesion of the exemplary hydrogels are desirable. For example, hydrogels according to one or more embodiments of the present disclosure may be useful in biomedical applications.

Exemplary hydrogels of the present disclosure showed great injectability (e.g. see FIG. 12 a and b) and strong adhesion to various substrates. Therefore, it is expected that hydrogel fibers may be produced accordingly, and such a process may be amenable to large-scale production. For example, a continuous spinning process could be realized by combining a syringe pump and a rotating collector to scalably fabricate hydrogel fibers (FIG. 13). In one example, a spinning process started with manually adhering a small volume of hydrogel to a rotating collector. Owing to the excellent stretchability and strong adhesion of the exemplary hydrogel, it adhered firmly to the collector. Subsequent stretching resulted in a fiber-like shape without fracture. It is suspected that, as the room humidity was relatively low (˜13%), the water in the as-generated hydrogel fiber would quickly evaporate during the exposure to ambient air, leading to a strengthened hydrogel fiber as compared to the fiber just coming out from the needle tip. After that, a fiber was collected by the rotating collector, which may act as a bridge to draw and stretch the hydrogel to generate a fiber, resulting in a continuous spinning process (e.g. see FIG. 30). The resulting hydrogel fibers showed a homogeneous diameter of ˜20 μm (FIG. 14) and retained strong mechanical properties (FIG. 15) as compared to manually fabricated hydrogel fibers, indicated by lifting a bottle (317.2 g) using piled hydrogel fibers (total diameter: 195.6±9.3 μm; total weight: 9.5 mg). Hydrogel fibers disclosed herein show stronger tensile stress and modulus than many reported bio-hydrogel fibers and soft tissues—including one of the toughest tissues, the tendon—demonstrating their potential use in biological and biomedical applications (e.g. see FIG. 16).

In summary, disclosed herein are examples of ultra-stretchable (over 12,400%) hydrogels with long-term adhesion, great injectability, and strong antimicrobial activity. Such hydrogels may be synthesized via oxidative decarboxylation between the interaction of Ag-lignin NPs and persulfate salt under room temperature. Such a process may be used for fabricating strong 1D macroscopic hydrogel fibers. The quinone-catechol reversible redox reaction and released Ag⁺ by Ag-lignin NPs may result in inherent antimicrobial activity and robust adhesion to various substrates. Hydrogel materials according to the present disclosure may be suitable for applications in tissue integration, repair, and replacement in biological and biomedical science. The mechanical properties of the disclosed hydrogels may be adjustable over a wide range, for example, by varying the dosages of P(AAm-co-AA) and CA in the precursor solution. Such variation in mechanical properties may make the resulting fiber widely adaptable for use in various tissues of different mechanical strength. In addition, the hydrogel fiber could be scalably produced via a facile spinning process. In one example, the fibers obtained as such exhibited a uniform diameter of ˜20 μm. By piling the spined fibers together, such fiber bundle of a diameter ˜200 μm exhibited excellent mechanical properties, indicated by lifting a load (317.9 g) of ˜33,000 times of its own weight (9.5 mg). Hydrogels according to the present disclosure may offer long-term adhesion, strong antimicrobial activity, excellent mechanical properties, and/or scalable production. It is expected that hydrogel fibers produced therefrom have potential for wound repair, tissue-mimicking and integration, and soft robotics in the fields of biological and biomedical science.

In one or more embodiments there is provided a composition comprising: a nanoparticle (NP) suspension; an organic acid, the organic acid having carboxylate groups; an oxidizing agent; acrylic acid (AA); and a polymerized copolymer containing AA.

### Nanoparticle Suspensions:

The NP suspension may be a silver-lignin nanoparticle (Ag-lignin NP) suspension. The NP may be a suspension of any suitable nanoparticle for generating free radicals, such as through an oxidative decarboxylation reaction. Therefore, it is expected that nanoparticles comprising silver, iron, copper, zinc, nickel, or combinations thereof, may be used. The nanoparticle suspension may be a suspension of nanoparticles selected from the group consisting of silver, iron, copper, zinc, nickel, and combinations thereof. The NP suspension may be a suspension of silver nanoparticles. The NP suspension may comprise a phenolic compound, such as catechol or lignin. The NP suspension may comprise polyphenols or polymers containing cross-linked phenolic rings. The NP suspension according to one or more embodiments comprises Ag-lignin NPs. Ag-lignin NPs may be obtained by a redox redox reaction between [Ag(NH₃)₂]⁺ and lignin aqueous solution. The NP suspension may be a source of persistent free radicals. The NP suspension may comprise a polyphenol and a free radical initiator. The NP suspension may comprise a nanoparticle containing catechol and a free radical initiator. The NP suspension may comprise silver and catechol. It will be understood that modifications of the NP suspension may result in modifications of the properties of the resultant hydrogel. In at least this way, the properties of hydrogels according to such embodiments may be variable, for example, their mechanical properties may be variable.

### Organic Acids:

The organic acid may be citric acid (CA). In one or more embodiments, the organic acid is an organic acid having carboxylate groups, such as three or more carboxylate groups. The organic acid may comprise any suitable compound for generating free radicals, such as through an oxidative decarboxylation reaction. Therefore, it is expected that any suitable carboxylate may be used. The organic acid may be citric acid, malic acid, or tartaric acid, or any combination thereof. The organic acid may comprise citric acid. Without wishing to be bound by any particular theory, it is suspected that the exemplified organic acid, citric acid, plays a role in enhancing the mechanical properties of the resulting hydrogel. Therefore, adjusting the concentration of organic acid in the composition or precursor solution may influence the mechanical properties of a resulting hydrogel or hydrogel fiber. At least in this way, the properties of the hydrogel according to one or more embodiments may have adjustable mechanical properties over a wide range. The amount or type of organic acid used may depend upon the application, such as the imitation of certain types of tissues, or the desired mechanical properties of the hydrogel fibers derived from the composition.

### Oxidizing Agents:

The oxidizing agent may be ammonium persulfate. In one or more embodiments, the oxidizing agent may comprise any suitable compound for facilitating the oxidative decarboxylation reaction, and/or regenerating any compound reduced in the process. The oxidizing agent may generate or assist in generating free radicals. The oxidizing agent may, directly or indirectly, generate a radical species which may add to an acrylic acid monomer to initiate the free radical polymerization of acrylic acid. The oxidizing agent may be ammonium persulfate, potassium persulfate, ferric solution, or hydrogen peroxide, or any combination thereof. The oxidizing agent may comprise a persulfate salt, such as ammonium persulfate.

### Polymerized Copolymers:

The polymerized copolymer may be poly(acrylamide-co-acrylic acid) (P(AAm-co-AA)). In one or more embodiments, the polymerized copolymer is a copolymer comprising acrylic acid (AA), or a polymer polymerized from acrylic acid and at least one additional monomer selected from acrylates, such as acrylamide. The polymerized copolymer may be any suitable polymer for adjusting the mechanical properties of the hydrogel. The polymerized copolymer may be poly(acrylamide-co-acrylic acid) (P(AAm-co-AA)), poly (ethylene glycol) dimethacrylate (PEGDA), or polyacrylic acid (PAA), or any combination thereof. The polymerized copolymer may be any suitable polymer comprising acrylates. The polymerized copolymer may comprise (P(AAm-co-AA)).

### Compositions:

In one or more embodiments, the composition may be an aqueous solution. The composition may be suspended or dissolved in any suitable solvent. The solvent may be an aqueous solution of basic or neutral pH. The solvent may be an aqueous solution of a pH suitable to prepare an Ag-lignin NP suspension. The composition may comprise other components. The composition or hydrogel according to one or more embodiments may have an odour, and an anti-odor agent may be added. Other additives include but are not limited to antimicrobial agents, antioxidants, plasticisers, colorants, conductive polymers (conductive) or metal/metal oxide nanoparticles (conductive), or any combination thereof.

The composition may comprise the NP suspension in an amount of from about 20 v/v % to about 60 v/v %, or about 25 v/v % to about 50 v/v %, or about 20 v/v % to about 40 v/v %, or about 20 v/v % to about 30 v/v %, or about 30 v/v % to about 40 v/v %, or about 30 v/v % to about 50 v/v %, or about 30 v/v % to about 60 v/v %, or about 35 v/v % to about 50 v/v %, or about 35 v/v % to about 45 v/v %, or about 40 v/v % to about 45 v/v %, or about 40 v/v % to about 50 v/v %, or about 40 v/v % to about 60 v/v %, or about 45 v/v % to about 50 v/v %, or about 45 v/v % to about 55 v/v %, or about 45 v/v % to about 60 v/v %, or about 50 v/v % to about 60 v/v %, such as about 20 v/v %, or about 25 v/v %, or about 30 v/v %, or about 35 v/v %, or about 36 v/v %, or about 37 v/v %, or about 38 v/v %, or about 39 v/v %, or about 40 v/v %, or about 41 v/v %, or about 41.6 v/v %, or about 42 v/v %, or about 43 v/v %, or about 44 v/v %, or about 45 v/v %, or about 50 v/v %, or about 55 v/v %, or about 60 v/v %. The composition may comprise the organic acid in an amount of from about 0.1 v/v % to about 5 v/v %. The composition may comprise the organic acid in an amount of from about 0.5 v/v % to about 2.5 v/v %, or about 1 v/v % to about 2 v/v %, or about 0.5 v/v % to about 1 v/v %, or about 1 v/v % to about 2.5 v/v %, or about 2 v/v % to about 2.5 v/v %, or about 1.5 v/v % to about 2.5 v/v %, such as about 0.5 v/v %, or about 1.0 v/v %, or about 1.5 v/v %, or about 2.0 v/v %, or about 2.5 v/v %.

The composition may comprise acrylic acid (AA) in an amount of from about v/v % to about 20 v/v %, or about 10 v/v % to about 12 v/v %, or about 10 v/v % to about 14 v/v %, or about 10 v/v % to about 15 v/v %, or about 10 v/v % to about 16 v/v %, or about 10 v/v % to about 18 v/v %, or about 12 v/v % to about 18 v/v %, or about 12 v/v % to about 16 v/v %, or about 12 v/v % to about 14 v/v %, or about 10 v/v % to about 12.5 v/v %, or about 12.5 v/v % to about 15 v/v %, or about 15 v/v % to about 17.5 v/v %, or about 12.5 v/v % to about 17.5 v/v %, or about 14 v/v % to about 16 v/v %, or about 14 v/v % to about 18 v/v %, or about 14 v/v % to about 20 v/v %, or about 15 v/v % to about 20 v/v %, or about 15 v/v % to about 17 v/v %, or about 15 v/v % to about 18 v/v %, or about 16 v/v % to about 18 v/v %, or about 16 v/v % to about 20 v/v %, or about 17 v/v % to about 20 v/v %, or about 18 v/v % to about 20 v/v %, such as about 10 v/v %, or about 12 v/v %, or about 14 v/v %, or about 15 v/v %, or about 16 v/v %, or about 18 v/v %, or about 20 v/v %.

The composition may comprise the polymerized copolymer in an amount of from about 0 w/v % to about 20 w/v %, or about 0.1 w/v % to about 20 w/v %, or about 0.1 w/v % to about 17 w/v %, or about 0 w/v % to about 17 w/v %, or about 0 w/v % to about 10 w/v %, or about 0.1 w/v % to about 10 w/v %, or about 1 w/v % to about 20 w/v %, or about 1 w/v % to about 15 w/v %, or about 1 w/v % to about 10 w/v %, or about 5 w/v % to about 20 w/v %, or about 5 w/v % to about 15 w/v %, or about 5 w/v % to about 10 w/v %, or about 10 w/v % to about 15 w/v %, or about 10 w/v % to about 20 w/v %, or about 15 w/v % to about 20 w/v %, or about 16 w/v % to about 18 w/v %, or about 16 w/v % to about 17 w/v %, such as about 0.1 w/v %, or about 0.5 w/v %, or about 1 w/v %, or about 2 w/v %, or about 2.5 w/v %, or about 5 w/v %, or about 10 w/v %, or about 15 w/v %, or about 16 w/v %, or about 16.6 w/v %, or about 17 w/v %, or about 18 w/v %, or about 19 w/v %, or about 20 w/v %.

### Hydrogels:

In one aspect there is provided a hydrogel obtained or obtainable by gelling the composition according to one or more embodiments of the present disclosure. The composition may be gelled using any suitable method or conditions. It will be understood that the gelling conditions may influence the properties of the resulting hydrogel. The composition may be gelled at room temperature. The composition may be gelled for any suitable amount of time. The hydrogel may be prepared by incubating the composition for an amount of time of about 8 hours to about 48 hours, or about 8 hours to about 16 hours, or about 8 hours to about 24 hours, or about 16 hours to about 24 hours, or about 16 hours to about 48 hours, or about 24 hours to about 48 hours, such as about 8 hours, about 12 hours, about 16 hours, about 20 hours, about 24 hours, about 30 hours, about 36 hours, about 40 hours, about 45 hours, or about 48 hours or more. It is considered advantageous that the hydrogels according to the present disclosure may be gelled from a composition at room temperature without requiring UV irradiation or heating. It will be understood that the compositions may be gelled in the presence of other stimulus, and by any suitable method, without departing from the spirit of the invention. Hydrogels may be obtained by gelling the composition in the absence of UV radiation or heating.

### Properties of the Hydrogels:

Hydrogels obtained by gelling the composition according to one or more embodiments of the present disclosure may have desirable mechanical properties. The hydrogel may have tensile stress in the range of about 97.6 MPa to about 422 MPa, or about 95 to about 425 MPa, or about 100 to about 425 MPa, or about 100 to about 400 MPa, or about 200 to about 400 MPa, or about 300 to about 400 MPa, such as about 100 MPa, or about 150 MPa, or about 200 MPa, or about 250 MPa, or about 300 MPa, or about 400 MPa, or about 420 MPa or more. The hydrogel may have strain in the range of about 6.7% to about 95.2%, or about 5% to about 95%, or about 10% to about 90%, or about 6.7% to about 10%, or about 90% to about 96%, or about 10% to about 25%, or about 25% to about 50%, or about 25% to about 75%, or about 25% to about 95%, or about 50% to about 75%, or about 50% to about 95%, or about 50% to about 90%, or about 75% to about 90%, or about 75% to about 95%, or about 80% to about 95%, such as about 5%, about 6.7%, about 10%, about 15%, about 20%, about 25%, about 30%, about 40%, about 50%, about 60%, about 70%, about 75%, about 80%, about 85%, about 90%, about 95%, or about 95.2%. The hydrogel may have Young's modulus in the range of about 1.2 GPa to about 8.7 GPa, or about 1 GPa to about 9 GPa, or about 1 GPa to about 8 GPa, or about 2 GPa to about 9 GPa, or about 2 GPa to about 8 GPa, or about 1 GPa to about 2 GPa, or about 8 GPa to about 9 GPa, or about 2 GPa to about 5 GPa, or about 5 GPa to about 8 GPa, or about 3 GPa to about 6 GPa, such as about 1 GPa, about 2 GPa, about 3 GPa, about 4 GPa, about 5 GPa, about 6 GPa, about 7 GPa, about 8 GPa, or about 9 GPa. The hydrogel may have toughness in the range of about 3.9 MJ m⁻³ to about 281.6 MJ m⁻³, or about 4 MJ m⁻³ to about 300 MJ m⁻³, or about 4 MJ m⁻³ to about 285 MJ m⁻³, or about 4 MJ m⁻³ to about 280 MJ m⁻³, or about 5 MJ m⁻³ to about 275 MJ m⁻³, or about 5 MJ m⁻³ to about 250 MJ m⁻³, or about 4 MJ m⁻³ to about 10 MJ m⁻³, or about 4 MJ m⁻³ to about 50 MJ m⁻³, or about 50 MJ m⁻³ to about 250 MJ m⁻³, or about 50 MJ m⁻³ to about 100 MJ m⁻³, or about 100 MJ m⁻³ to about 200 MJ m⁻³, or about 200 MJ m⁻³ to about 285 MJ m⁻³, or about 250 MJ m⁻³ to about 285 MJ m⁻³, or about 250 MJ m⁻³ to about 275 MJ m⁻³, or about 260 MJ m⁻³ to about 280 MJ m⁻³, such as about 3.9 MJ m⁻³, or about 4 MJ m⁻³, or about 5 MJ m⁻³, or about 10 MJ m⁻³, or about 25 MJ m⁻³, or about 50 MJ m⁻³, or about 100 MJ m⁻³, or about 150 MJ m⁻³, or about 200 MJ m⁻³, or about 250 MJ m⁻³, or about 275 MJ m⁻³, or about 280 MJ m⁻³, or about 281.6 MJ m⁻³, or about 285 MJ m⁻³.

The hydrogel may have antimicrobial activity such as antibacterial activity. The hydrogel may have low or substantially no cytotoxicity. The hydrogel may be a hydrogel fiber. The hydrogel may be stretched or spun to form a hydrogel fiber. The hydrogel fiber may be micro-sized. The hydrogel fiber may have a diameter of about 20 μm to about 150 μm, or about 25 to about 50 μm, or about 50 to about 100 μm, or about 100 to about 150 μm, such as less than about 20 μm, about 25 μm, about 50 μm, about 75 μm, about 100 μm, about 125 μm, about 150 μm or more.

### Applications and Uses:

Hydrogels according to one or more embodiments of the present disclosure, or gelled from compositions according to one or more embodiments or the present disclosure, may be used for any suitable application, such as load-bearing applications. The hydrogels may be used as adhesives or coatings. The hydrogel may be for adhesion to, or to coat: mammalian tissue such as skin, organs, bone, kidney, heart, lung, or liver; plant or plant materials such as fruits or vegetables; or other materials such as polymers, rubbers, metals, fibers, or clothing such as masks. The biocompatibility of such hydrogels suggests they may be desirable for use in biomedical applications. The hydrogels or hydrogel fibers may be used in biomedical applications or medical devices, such as implants, wound repair (sutures, bandages, coatings), or artificial tissues. The hydrogel or hydrogel fibers may be used as a coating for any suitable material, such as a mask, a bandage, or other medical device. In one aspect, there is provided an artificial tissue comprising the hydrogel according to one or more embodiments. The artificial tissue may be any suitable tissue, such as one having mechanical properties similar to that of the hydrogel. For example, the artificial tissue may be an artificial muscle, tendon, or cartilage. The properties of the hydrogel may be tuned depending upon the application or use. While biomedical applications are mentioned herein, it will be understood that the hydrogels of the present disclosure may have favourable mechanical properties for any number of non-medical uses.

### Methods:

In one aspect there is provided a method of preparing a hydrogel comprising: combining the components of the hydrogel precursor solution and incubating the precursor solution to form the hydrogel. The method may comprise combining nanoparticles, an organic acid, an oxidizing agent, acrylic acid, and a polymerized copolymer containing AA. For example, the method may comprise combining Ag-lignin NPs, citric acid, a persulfate salt, acrylic acid, and (P(AAm-co-AA)). Precursor solutions or compositions herein disclosed may be used in the method. The method may include incubating the hydrogel precursor solution at room temperature for any suitable amount of time, such as about 8 hours to about 48 hours. The method may include preparing or obtaining Ag-lignin NPs by any suitable method, such as by a redox reaction between [Ag(NH₃)₂]⁺ and lignin aqueous solution. The method may include any number of additional steps, such as steps to shape, form, or otherwise modify the resultant hydrogel. The method may include stretching the gelled hydrogel to form a hydrogel fiber. Stretching the hydrogel to form a hydrogel fiber may include a spinning or stretching process. The method may include agitation of the precursor solution, such as mechanical agitation or sonication. It will be understood that the method of preparing a hydrogel may be tuned to a particular application, or to achieve specific mechanical properties, and may be variable. In one aspect there is provided an apparatus for preparing a hydrogel fiber, the apparatus comprising: a pump for dispensing a solution of the hydrogel to form the hydrogel fiber and a collector for receiving the hydrogel fiber.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

Materials. Poly(acrylamide-co-acrylic acid) (P(AAm-co-AA), Mw:˜520,000, Mn:˜150,000, acrylamide ˜80 wt. %), silver nitrate (AgNO₃, ReagentPlus®, ≥99.0%), lignin alkali (average Mw ˜10,000, low sulfonate content), citric acid (ACS reagent, ≥99.5%), and ammonium hydroxide solution (ACS reagent, 28.0-30.0%, NH₃ basis) were purchased from Sigma-Aldrich. Sodium hydroxide (Certified ACS) was received from Fisher Chemical, acrylic acid (AA, anhydrous, contains 200 ppm MEHQ as inhibitor, 99%) was purchased from Aldrich, and ammonium persulfate (APS, for molecular biology, for electrophores, ≥98%) was bought from Sigma. All the chemicals were received without further purification. The deionized (DI) water (resistivity ≥18.2 MΩ·cm) was used whenever needed.

Preparation of Ag-Lignin Nanoparticles (NPs). The Ag-lignin NPs were synthesized according to the reported methods with some modifications. An aqueous lignin solution (20 mg/mL) was prepared by dissolving lignin of low sulfonate content (0.4 g) in DI water (20 mL) assisted by sonication for 1 h. The resulting solution was named solution A. In the meantime, another solution (solution B) was prepared by dissolving AgNO₃ (126 mg) in DI water (8 mL), followed by mixing with ammonium hydroxide solution (5 mol/L, 2 mL). After that, solution A was poured into solution B and stirred at 300 rpm at room temperature for 24 h. The final solution was denoted as Ag-lignin NPs solution. To obtain dry Ag-lignin NPs, the corresponding solution was frozen at −28° C. overnight and freeze-dried using a freeze drier (LABCONCO, FreeZone 2.5 L—50° C. Benchtop).

Preparation of Citric Acid/Water Solution. The citric acid (CA) aqueous solution (10 w/v %) was prepared by dissolving CA (2 g) in DI water (20 mL), followed by adding NaOH (1.2 g) to neutralize the CA solution. In this work, CA solution with different concentrations of weight/volume percentage (2.5 w/v %, 5 w/v %, 7.5 w/v % or 10 w/v %) was prepared and used for synthesizing the hydrogel.

Preparation of Ag-Lignin NPs Based Hydrogel. P(AAm-co-AA) (0.5 g), AA (1 mL), CA solution (5 w/v %, 1.5 mL), freshly prepared Ag-lignin NPs solution (2.5 mL), and APS/water solution (1 wt. %, 1 mL) were mixed together and transported to a centrifuge tube (50 mL), followed by vigorous shaking using a vortex mixer (VWR International) at the maximum output level for 60 s. The mixture obtained as such was then incubated at room temperature for 24 h. The resulting hydrogel was named 0.5P-1AA-5CA hydrogel. All the synthesized hydrogels in this work are denoted in the form as “aP-bAA-cCA”, where “a” represents the mass of added P(AAm-co-AA), “b” represents the volume (mL) of AA in the precursor solution, and “c” represents the weight percentage of CA in the corresponding CA/water solution. The abbreviations of synthesized hydrogel and the dosage of materials used for each entry in this work are shown in Table S1.

Manual Fabrication of Hydrogel Fiber. To fabricate a hydrogel fiber with straight shape and uniform diameter, a small amount of hydrogel was attached to the tip of the index finger, followed by adhesion on another index finger. The hydrogel fiber was formed by separating two fingers at a speed of about 4 cm s⁻¹, and the fiber obtained as such was then exposed to the ambient air (temperature: ˜23.5° C., room humidity: ˜13%) for 5 min prior to subsequent characterizations.

Characterization of Hydrogels and Generated Hydrogel Fibers. The microscopic structure of hydrogels or generated fibers was characterized using an FE-SEM (Zeiss Sigma) or an inverted microscope (Olympus BX 73). Transmission electron microscopy (TEM) images of the Ag-lignin NPs were taken with a JEOL JEM-ARM200CF S/TEM electron microscope at an accelerating voltage of 200 kV. The high-angle annular dark-field scanning transmission electron microscopy (HAADF-STEM) images were collected with the JEOL HAADF detector with the following experimental conditions: condenser lens aperture of ˜30 μm, scan speed of ˜32 us pixel⁻¹, and camera length of ˜8 cm. TEM samples were prepared by depositing a droplet of suspension of Ag-lignin NPs in DI water onto an ultra-thin carbon-coated copper grid (Electron Microscopy Science Inc.), and the grid obtained as such was dried at least 24 h prior to data collection. The patterns of lignin NPs and freeze-dried Ag-lignin NPs were obtained using an X-ray diffraction instrument (Rigaku Multiflex) with Cu Kα X-ray radiation. The FTIR spectrometer (NICOLET iS20, Thermo Scientific) with an ATR sampling accessory was used to determine the spectra of hydrogels, hydrogel fibers, Ag-lignin particles, and raw materials, including P(AAm-co-AA) and AA.

Tensile Test. An electronic universal testing machine (Shenzhen Suns Technology Stock Co, LTD.) was used to determine the tensile stress-strain curves of the hydrogels and the fabricated hydrogel fibers. To obtain the stress-strain curve of the fiber, both ends of the fiber were first clamped by the crossheads with an initial distance of 15 mm. After that, the upper crosshead was moved upwards at a speed of 5 mm min-1 until the breaking of the fiber. The diameter of all the tested hydrogel fibers was 33±8 μm.

To determine the tensile stress-strain curves of the hydrogels (Table 1, Entries 3b-3d), the hydrogel solution before gelation was first transferred into a 1 mL syringe (Thermo Scientific™, Luer-Slip Disposable Syringe). After 24 h, the gelled solution was injected out from the syringe, and both ends of the hydrogel were clamped by the crossheads (the initial distance between the crossheads was 15 mm for Entries 3c and 3d, and 5 mm for Entry 3b). Then, the crosshead on the top was moved upwards at a speed of 50 mm min 1 until the breaking of the hydrogel or reaching the operation limit of the machine.

Antimicrobial Activity. The Gram-negative Escherichia coli, BL21(DE3) was used for the experiments. A monoclony of E. coli was inoculated in Luria-Bertani (LB) broth overnight at 37° ° C. with shaking (220 rpm). After that, 1 ml overnight E. coli culture was added to 50 ml fresh LB broth, continuing culturing for 1.5 h with shaking (220 rpm). The resulting E. coli culture (˜2×108 CFU/mL) was aliquoted to test tubes for the antimicrobial assays, and the studied hydrogel (50 mg) was then added to the prepared E. coli culture (5 ml), followed by incubation at 37° C. for 24 h with a shaking speed of 100 rpm. An aliquot of 100 μL was then taken out from the mixture to determine the corresponding colony-forming unit (CFU) by doing serial dilutions and plating on LB agar plates.

UV-vis Spetra Measurement of Ag-lignin and lignin NPs. The UV-vis spectra of the suspension of Ag-lignin and lignin NPs (0.2 mg/mL) in DI water were recorded in the region from 250 nm to 800 nm using a UV-vis spectrophotometer (Cary 3500, Agilent) with a resolution of 1 nm. The DI water was used as the background. The surface plasmon resonance of Ag-lignin NPs was indicated by the increased absorbency in the region between 400 nm-500 nm with the presence of Ag NPs.

### Calculation of Tensile Stress, Strain, Young's Modulus and Toughness.

The tensile stress (σ) of the generated hydrogel fiber was calculated using Equation (1):

$$σ = \frac{F}{π {r}^{2}} \qquad ( 1 )$$

where F is the load force recorded by the universal machine and r is the initial radius of the generated hydrogel fiber. The initial radius was measured using a micrometer at three different positions, and the average value is used as r.

The tensile strain (8) of the hydrogel fiber was calculated using the following Equation (2):

$$ε = \frac{l - {l}_{0}}{{l}_{0}} \qquad ( 2 )$$

where l₀ is the initial length of the hydrogel fiber between the crossheads, and l is the length of the fiber right before the rupture. In the tensile test, all the initial lengths of the generated fibers were set as 15 mm.

The toughness (T) is an engineering value used for evaluating the ability of a material to absorb energy and plastically deform without fracturing. The T of the generated hydrogel fiber was calculated using Equation (3):

$$T = {\int }_{0}^{ε} σ d ε \qquad ( 3 )$$

The Young's modulus (E) is used to evaluate the tensile stiffness of the hydrogel fiber and was calculated using Equation (4):

$$E = \frac{σ}{ε} \qquad ( 4 )$$

Scalable Production of Hydrogel Fibers by Spinning. The freshly prepared precursor solution was mixed together with 1 mL 1 wt. % APS aqueous solution, followed by loading into a 1 ml syringe (Thermo Scientific™, Luer-Slip Disposable Syringe). After 8h of gelling reaction, a needle (Needle Gauge 19, outer diameter: 1.07 mm and inner diameter: 0.69 mm) was connected to the syringe prior to the following spinning process. With the syringe pump (Masterflex Touch-Screen Syringe Pump, Infusion, Two-Syringe) turned on, a small tip of injectable hydrogel was manually drawn into a fiber and then adhere to the rotating collector (Milefo Cup Turner Spinner, CUPTURNER). The spinning process was done at room temperature of ˜21.7° C. and room humidity of ˜13%. The hydrogel was infused at a speed of 0.0005 mL/min and the collector with a diameter of ˜95 mm rotated at a speed of 5-6 rpm.

TABLE 1
Screening studies of design of highly stretchable, antimicrobial, and adhesive hydrogels
for engineering hydrogel fibers (Ag-lignin solution of 2.5 mL and 1 wt. % APS solution
were mixed with all the entries to proceed possible gelation process).

| | | | | | Wire | |
|---|---|---|---|---|---|---|
| | P(AAm-co-AA) | AA | CA/Water | | Drawing | |
| Entry | (g) | (mL) | (w/v %, mL) | Hydrogel | Ability ^{a} | Abbreviation |
| 1a | 0.5 | 0 | 0 w/v %, | x | — | — |
| | | | 1.5 mL | | | |
| 1b | 0 | 1 | 0 w/v %, | ✓ | x | — |
| | | | 1.5 mL | | | |
| 1c | 0 | 0 | 10 w/v %, | x | — | — |
| | | | 1.5 mL | | | |
| 2a | 0.5 | 0 | 10 w/v %, | x | — | 0.5P-0AA-10CA |
| | | | 1.5 mL | | | |
| 2b | 0.5 | 1 | 0 w/v %, | ✓ | ✓ | 0.5P-1AA-0CA |
| | | | 1.5 mL | | | |
| 2c | 0 | 1 | 10 w/v %, | ✓ | ✓ | 0P-1AA-10CA |
| | | | 1.5 mL | | | |
| 3a | 0.5 | 0.5 | 2.5 w/v %, | x | — | 0.5P-0.5AA-2.5CA |
| | | | 2.0 mL | | | |
| 3b | 0.5 | 1 | 2.5 w/v %, | ✓ | ✓ | 0.5P-1AA-2.5CA |
| | | | 1.5 mL | | | |
| 3c | 0.5 | 1.5 | 2.5 w/v %, | ✓ | x | 0.5P-1.5AA-2.5CA |
| | | | 1.0 mL | | | |
| 3d | 0.5 | 2.0 | 2.5 w/v %, | ✓ | x | 0.5P-2AA-2.5CA |
| | | | 0.5 mL | | | |
| 4a | 0.5 | 1 | 5 w/v %, | ✓ | ✓ | 0.5P-1AA-5CA |
| | | | 1.5 mL | | | |
| 4b | 0.5 | 1 | 7.5 w/v %, | ✓ | ✓ | 0.5P-1AA-7.5CA |
| | | | 1.5 mL | | | |
| 4c | 0.5 | 1 | 10 w/v %, | ✓ | ✓ | 0.5P-1AA-10CA |
| | | | 1.5 mL | | | |
| 5a | 0 | 1 | 10 w/v %, | ✓ | ✓ | 0P-1AA-10CA |
| | | | 1.5 mL | | | |
| 5b | 0.25 | 1 | 10 w/v %, | ✓ | ✓ | 0.25P-1AA-10CA |
| | | | 1.5 mL | | | |
| 5c | 0.75 | 1 | 10 w/v %, | ✓ | ✓ | 0.75P-1AA-10CA |
| | | | 1.5 mL | | | |
| 5d | 1 | 1 | 10 w/v %, | ✓ | ✓ | 1P-1AA-10CA |
| | | | 1.5 mL | | | |

^{a} Wire drawing ability of the hydrogel was determined by whether the hydrogel could be stretched to corresponding fiber with a diameter of ~50 μm.

The embodiments described herein are intended to be examples only. Alterations, modifications, and variations can be effected to the particular embodiments by those of skill in the art. The scope of the claims should not be limited by the particular embodiments set forth herein but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A composition comprising:
a nanoparticle (NP) suspension;
an organic acid, the organic acid having carboxylate groups;
an oxidizing agent;
acrylic acid (AA); and
a polymerized copolymer containing AA.

2. The composition of claim 1, wherein the NP suspension is a suspension of nanoparticles selected from the group consisting of silver, iron, copper, zinc, nickel, and combinations thereof.

3. The composition of claim 1 or 2, wherein the NP suspension is a suspension of silver nanoparticles.

4. The composition of any one of claims 1 to 3, wherein the NP suspension comprises a phenolic compound.

5. The composition of claim 4, wherein the phenolic compound comprises catechol.

6. The composition of any one of claims 1 to 5, wherein the NP suspension comprises Ag-lignin NPs.

7. The composition of any one of claims 1 to 6, wherein the organic acid is selected from the group consisting of citric acid, malic acid, tartaric acid, and combinations thereof.

8. The composition of any one of claims 1 to 7, wherein the organic acid comprises citric acid (CA).

9. The composition of any one of claims 1 to 8, wherein the polymerized copolymer is selected from the group consisting of poly(acrylamide-co-acrylic acid) (P(AAm-co-AA)), poly (ethylene glycol) dimethacrylate (PEGDA), polyacrylic acid (PAA), and combinations thereof.

10. The composition of any one of claims 1 to 9, wherein the polymerized copolymer comprises poly(acrylamide-co-acrylic acid) (P(AAm-co-AA)).

11. The composition of any one of claims 1 to 10, wherein the composition is an aqueous solution.

12. The composition of any one of claims 1 to 11, wherein the oxidizing agent is selected from the group consisting of ammonium persulfate, potassium persulfate, hydrogen peroxide, a ferric solution, and combinations thereof.

13. The composition of any one of claims 1 to 12, wherein the oxidizing agent comprises ammonium persulfate.

14. The composition of any one of claims 1 to 13, wherein the NP suspension is an Ag-lignin NP suspension obtained by a redox reaction between [Ag(NH₃)₂]⁺ and lignin aqueous solution.

15. The composition of any one of claims 1 to 14, wherein the composition comprises the NP suspension in an amount of about 20 v/v % to about 60 v/v %, such as about 40 v/v %.

16. The composition of any one of claims 1 to 15, wherein the composition comprises the organic acid in an amount of from about 0.5 v/v % to about 2.5 v/v %, such as about 2.0 v/v %.

17. The composition of any one of claims 1 to 16, wherein the composition comprises the AA in an amount of about 10 v/v % to about 20 v/v %, such as about 15 v/v %.

18. The composition of any one of claims 1 to 17, wherein the composition comprises the polymerized copolymer in an amount of from about 0.1 w/v % to about 20 w/v %, such as about 10 w/v %.

19. The composition of any one of claims 1 to 18 further comprising an additive selected from the group consisting of anti-odor agents, antimicrobial agents, antioxidants, plasticisers, colorants, other polymers such as conductive polymers, metal/metal oxide nanoparticles, or any combination thereof.

20. A hydrogel obtained by gelling the composition according to any one of claims 1 to 19.

21. The hydrogel of claim 20, wherein the hydrogel is obtained by incubating the composition at room temperature for an amount of time of about 8 hours to about 48 hours, or about 16 hour to about 24 hours, such as about 8 hours, about 16 hours, or about 24 hours.

22. The hydrogel of claim 20 or 21, wherein the hydrogel is obtained by gelling the composition in the absence of UV radiation or heating.

23. The hydrogel of any one of claims 20 to 22, wherein the hydrogel has tensile stress in the range of about 97.6 MPa to about 422.0 MPa.

24. The hydrogel of any one of claims 20 to 23, wherein the hydrogel has strain in the range of about 6.7% to about 95.2%.

25. The hydrogel of any one of claims 20 to 24, wherein the hydrogel has Young's modulus in the range of about 1.2 GPa to about 8.7 GPa.

26. The hydrogel of any one of claims 20 to 25, wherein the hydrogel has toughness in the range of about 3.9 MJ m⁻³ to about 281.6 MJ m⁻³.

27. The hydrogel of any one of claims 20 to 26, wherein the hydrogel has antimicrobial activity, such as antibacterial activity.

28. The hydrogel of any one of claims 20 to 27, wherein the hydrogel is a hydrogel fiber.

29. The hydrogel of claim 28, wherein the hydrogel fiber is micro-sized, having a diameter in the range of about 20 μm to about 150 μm.

30. Use of the hydrogel according to any one of claims 20 to 29 for bearing a load.

31. Use of the hydrogel according to any one of claims 20 to 29 as an adhesive or a coating.

32. The use of claim 31, wherein the hydrogel is for adhesion to, or to coat: mammalian tissue such as skin, organs, bone, kidney, heart, lung, or liver; plant or plant materials such as fruits or vegetables; or other materials such as polymers, rubbers, metals, fibers, or clothing such as masks.

33. Use of the hydrogel according to any one of claims 20 to 29 in a biomedical application or device.

34. The use of claim 33, wherein the biomedical application or device is selected from the group consisting of medical devices, implants, wound repair, sutures, bandages, coatings, or artificial tissues.

35. An artificial tissue comprising the hydrogel according to any one of claims 20 to 29.

36. The artificial tissue of claim 35, wherein the tissue is muscle, tendon, or cartilage.

37. A method of preparing a hydrogel comprising:
combining nanoparticles, an organic acid, an oxidizing agent, acrylic acid (AA), and a polymerized copolymer containing AA, in an aqueous solution to form a hydrogel precursor solution; and
incubating the hydrogel precursor solution to form the hydrogel.

38. The method of claim 37, wherein the nanoparticles comprise silver, iron, copper, zinc, or nickel nanoparticles.

39. The method of claim 37 or 38, wherein the nanoparticles comprise a suspension of Ag-lignin NPs.

40. The method of any one of claims 37 to 39, wherein the organic acid comprises citric acid.

41. The method of any one of claims 37 to 40, wherein the oxidizing agent comprises a persulfate salt.

42. The method of any one of claims 37 to 41, wherein the polymerized copolymer comprises poly(acrylamide-co-acrylic acid) (P(AAm-co-AA)).

43. The method of any one of claims 37 to 42, wherein the combining step comprises combining the polymerized copolymer with one or more solutions comprising the AA, the organic acid, the nanoparticles, the oxidizing agent, or any combination thereof.

44. The method of any one of claims 37 to 43, wherein incubating the hydrogel precursor solution comprises incubating at room temperature.

45. The method of any one of claims 37 to 44, wherein incubating the hydrogel precursor solution comprises incubating for an amount of time in the range from about 8 hours to about 48 hours.

46. The method of any one of claims 37 to 45, wherein the method further comprises preparing Ag-lignin NPs prior to the combining step.

47. The method of any one of claims 37 to 46, further comprising stretching the formed hydrogel to form a hydrogel fiber.

48. The method of claim 47, wherein stretching the formed hydrogel comprises a spinning process.

49. The method of any one of claims 37 to 48, wherein the method further comprises agitation of the precursor solution, such as mechanical agitation or sonication.

50. The method of any one of claims 37 to 49, wherein the incubating step is in the absence of UV radiation or heating.
